(19) United States
(12) Patent Application Publication
(10) Pub. No.: US 2004/0244226 A1
(43) Pub. Date: Dec. 9, 2004

(54) Article of footwear, particularly for climbing

(75) Inventors: Yves Farys, Saint-Jorioz (FR); Frederic Giacobone, Cusy (FR)

Correspondence Address: GREENBLUM & BERNSTEIN, P.L.C., 1950 ROLAND CLARKE PLACE, RESTON, VA, 20191, US

(73) Assignee: SALOMON S.A., Metz-Tessy (FR)

(21) Appl. No.: 10/718,553

(22) Filed: Nov. 24, 2003

(30) Foreign Application Priority Data
Dec. 11, 2002 (FR) 02.16238

(51) Int. Cl. A61F 5/14; A43B 23/10

(52) U.S. Cl. 36/140; 36/69

(57) ABSTRACT

An article of footwear, in particular for climbing, having a flexible upper provided with a tightening arrangement and an outer sole, with the upper having a molded heel stiffener. The assembly method includes:

assembling the upper to an insole;

pressing and gluing to the upper a molded heel stiffener;

pressing and gluing to the front of the upper a front protective strip;

pressing and gluing to the rear of the upper a strap overlapping the heel stiffener and the front protective strip;

pressing and gluing the outer sole.

### CROSS-REFERENCE TO RELATED APPLICATION

This application is based upon French Patent Application No. 02.16238, filed Dec. 11, 2002, the disclosure of which is hereby incorporated by reference thereto in its entirety and the priority of which is hereby claimed under 35 U.S.C. §119.

### BACKGROUND OF THE INVENTION

1. Field of the Invention

The invention relates to an article of footwear, particularly a slipper or ballerina type article, more specifically provided for climbing mountains, cliffs, ice, rocks, and artificial structures.

2. Description of Background and Relevant Information

The climber's shoe is a fundamental element of the climber's equipment. Indeed, the shoe is what is interposed between the climber's foot and the surface being climbed, and which must transmit all of the sensations/feelings and forces as precisely as possible to the climber.

A climbing shoe is constituted of a flexible upper, generally low or semi-high, made of a flexible and inextensible material, and provided at its upper end with a system for retaining the foot, such as a lace.

The upper is generally coated over its entire lower peripheral portion with a coating strip, also called a “strapping,” made of an adhesive material, such as rubber, and is provided in the area of the heel with a strap or stay made of an elastic material, such as rubber, that surrounds the heel obliquely downward from the rear of the heel to the front thereof, and applies a tension on the upper. Finally, a sole, made of rubber or the like and cemented to the upper, completes the assembly.

Known climbing shoes or slippers therefore have a large number of elements, particularly made of rubber, that are in the form of strips, and which are applied by adhesive/cementing and manually shaped according to the form of the upper. Consequently, the manufacturing cycle proves to be relatively long and costly.

Additionally, the reproducibility of the articles of footwear is not very good due to the high number of manual operations and lack of pre-shaping of the rubber strips applied. There results problems in comfort related to the absence of uniformity from one shoe to the next, particularly in the volume of the heel.

In order to overcome these drawbacks partially, European Patent Publication EP 688 512 and French Patent Publication FR 2 720 235 have proposed, for example, to make the strap or the stay and the outer sole in a single piece.

In the case of EP 688 512, the strap and the sole are formed together by molding. In addition, the strap completely envelopes the heel and therefore also constitutes a heel stiffener.

The drawback to this technology is that it is very expensive since a mold is necessary for each size. Also, it is difficult to resole the shoe once the sole is worn out since the heel risks being damaged.

The French Patent Publication FR 2 720 235 also proposes making a heel-stay-sole stiffener in a single piece. In the case of this document, the sole and the reinforcement (or stay or heel stiffener) are made from a single piece of a layered material, shaped by cementing to the upper.

This technology has the same disadvantage as the preceding with regards to repair. Additionally, the heel portion is manually shaped on the shoe and is subject to the same reproducibility drawbacks that were previously described.

### SUMMARY OF THE INVENTION

An object of the present invention is to overcome the aforementioned drawbacks and, in particular, to provide a new principle of construction that allows for a good reproducibility and uniformity of the articles of footwear. One of the objects is also to allow for greater ease in repair.

This object is achieved in the article of footwear according to the invention, particularly for climbing, which is of the type having a flexible upper provided with tightening, means and an outer sole, the upper having a molded heel stiffener.

Indeed, the fact that the stiffener is molded guarantees a predetermined and uniform fitting volume for the heel from one article of footwear to the next, and therefore resolves the problems in comfort and manufacturing homogeneity mentioned hereinabove.

### BRIEF DESCRIPTION OF DRAWINGS

The invention will be better understood and other characteristics thereof will become apparent by means of the following description, with reference to the attached schematic drawings that show, by way of non-limiting example, a preferred embodiment, and in which:

FIG. 1 is an exploded perspective view of a foot article according to the invention before assembly;

FIG. 2 is a front perspective view of the stiffener;

FIG. 3 is a rear perspective view of the stiffener;

FIG. 4 is a front perspective view of the article of footwear of FIG. 1 after assembly.

### DETAILED DESCRIPTION OF THE INVENTION

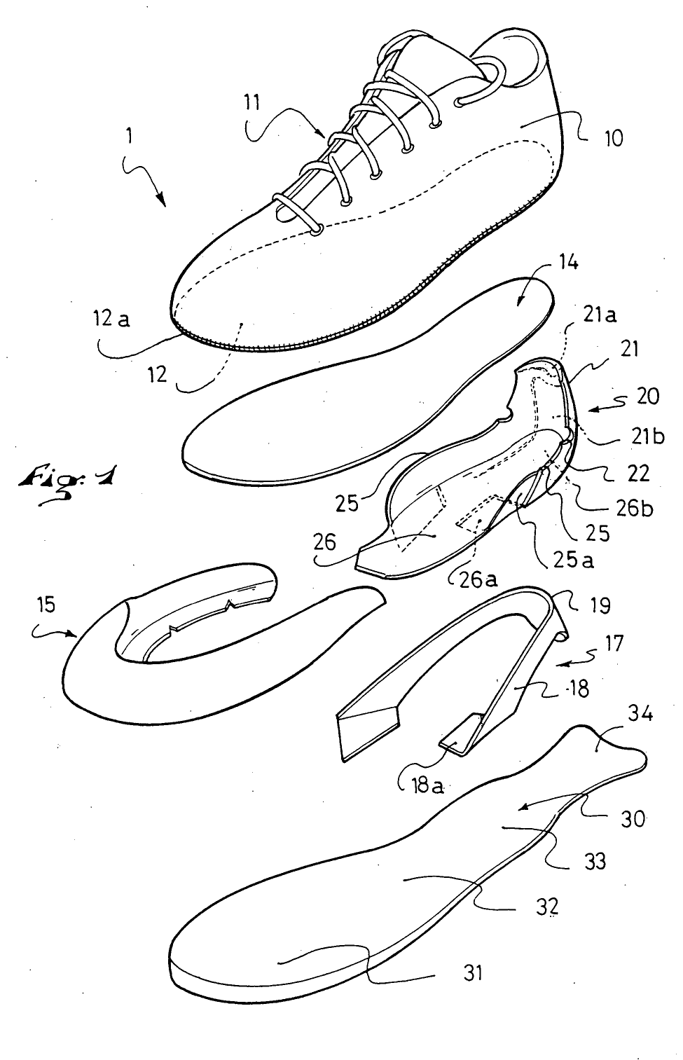

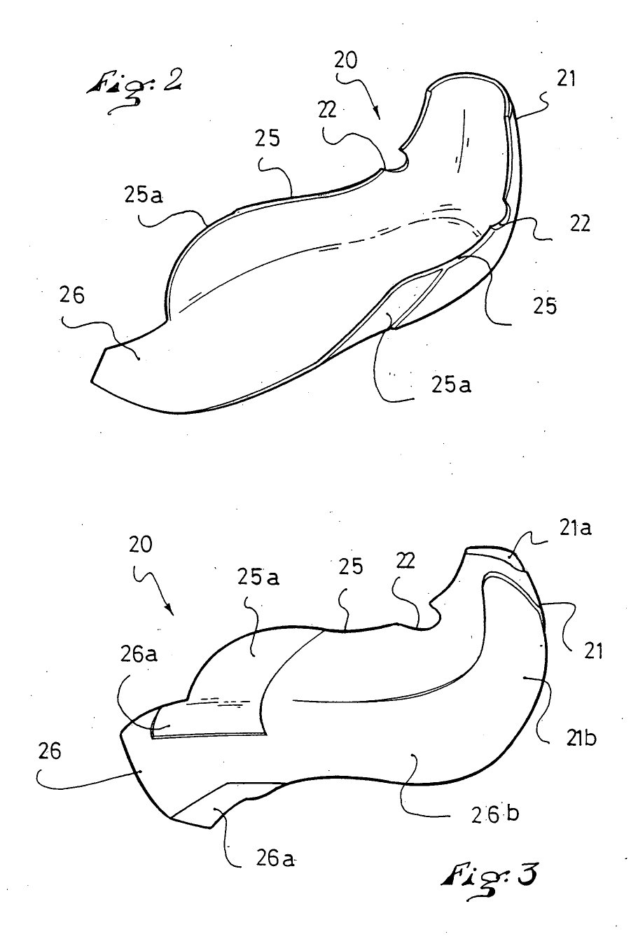

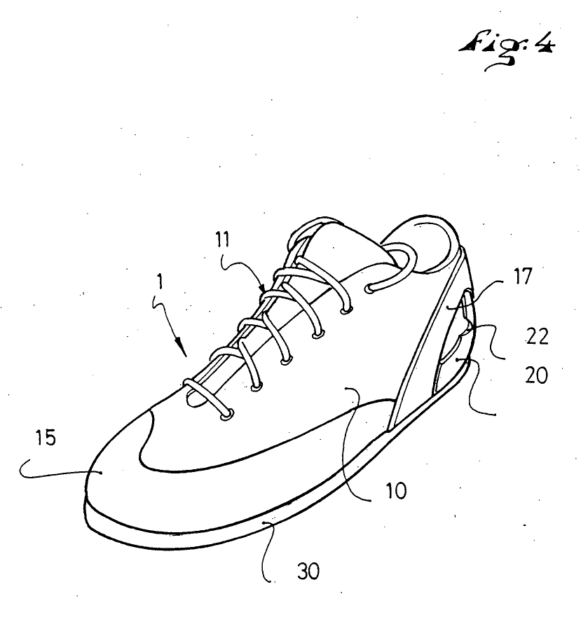

As shown in FIG. 1, the article of footwear or climbing shoe 1, according to the invention, has:

an upper 10 made of a flexible material, such as leather or a substantially inextensible textile, having a low or semi-high configuration as shown on the drawing, and provided with tightening means 11 of the lace type or the like;

an insole 12 peripherally assembled to the upper 10 by a seam 12a of the Strobel type or zigzag type, the insole 12 being flexible;

a reinforcement insert 14 extending substantially over the entire surface of the insole 12 and interposed between the latter and the outer sole, the object of this insert 14 being to help control as best as possible the deformations of the bottom assembly of the shoe during supports. Depending on the characteristics sought for the climbing shoe, this insert 14 can be more or less rigid; it can also be omitted. This type of construction with the insert is described in the French Patent Application FR 02.06811 and U.S. patent application Ser. No. 10/259,826, both commonly owned herewith, the latter being incorporated by reference thereto in its entirety particularly for its disclosure of the aforementioned insert;

a heel stiffener 20 and an outer sole 30 that are described more specifically hereinafter;

a front protective peripheral strip 15 applied to the front of the upper 10 and a strap or stay 17 surrounding the heel portion of the upper 10, and having two arms 18 and a rear portion 19.

The heel stiffener 20 is made by molding a flexible and adhesive material, such as rubber.

Preferably, the material used is a very adhesive rubber having a hardness of approximately 70 Shore A.

The stiffener 20 has a rear wall 21 that is substantially vertical, two lateral walls or edges 25, and a base 26. It is extended forwardly by its base 26 substantially up to the front limit of the zone of the plantar arch, and therefore to the rear of the metatarsophalangeal articulation zone. The lateral edges 25 stop substantially at the rear of the zone of the plantar arch. The rear wall 21, in particular, but also the lateral edges 25, together define a rounded volume corresponding as best as possible to the form of the heel and adapted to receive and comfortably house the wearer's heel. The lateral edges 25 act to protect the rear lateral edges of the upper in the same manner as the front peripheral protective strip 15.

At the junction between its rear wall 21 and the lateral edges 25, the heel stiffener has a notch 22 adapted to allow for the relative “pivoting” of the rear portion relative to these lateral edges when in use, and specifically when putting on the shoe.

As a matter of fact, the rear wall 21 has a very nesting rounded shape adapted to properly envelop the heel, and the strap 17 reinforces this effect since it acts to exert a thrusting effort of the heel toward the front. When putting the shoe on, it is necessary to “pivot” the wall 21 of the heel stiffener relative to the lateral edges 25 thereof in order to actually allow for the insertion of the foot.

The heel stiffener 20 also has recesses or housings adapted to facilitate the positioning and the cementing of the pieces that are applied next, namely the strap 17 and the outer sole 30. Primarily, those are recesses 25a at the front of each lateral edge and 26a underneath the base 26 for receiving the lower ends 18a of the arms 18 of the strap 17. It is possible for another recess 21a to be provided in the rear wall 21 to receive the rear portion 19 of the strap 17.

The recesses can be provided only for one of the elements, the strap or the outer sole, mentioned hereinabove.

Finally, in the rear wall 21 and in the base 26 of the stiffener, recesses 21b, 26b, respectively, are provided to receive an associated portion 34 of the sole 30,

As shown more particularly in FIG. 1, the sole 30 has three portions:

a first front portion 31 located essentially opposite the front zone of the shoe, with a thickness of 4 millimeters (mm);

an intermediary zone 32 located in the area of the plantar arch, and whose thickness decreases progressively from 4 to 2 mm;

a rear zone 33-34, with a thickness of 2 mm, a first section 33 therefore being adapted to cover the base 26 of the heel reinforcement 20, and a second section 34 adapted to rise along the rear wall 21 of the reinforcement.

The shoe according to the invention is assembled in a simple manner:

first of all, the reinforcement insert 14 is cemented or glued to the entire lower surface of the insole 12. As indicated previously, this reinforcement insert 14 can be present or not depending on the characteristics sought for the liner;

next, the heel stiffener 20 is pressed and cemented to the upper 10 and to its insole 12 or insert 14;

the front protective strip 15 is pressed and cemented to the front of the upper and to the periphery of its insole 12 or insert 14;

the strap or stay 17 is then positioned and cemented to the upper 10 and to the lasting allowance by the end 18a of its arms 18.

It is noted that the positioning of the strap on the upper is facilitated by the housings provided in the heel reinforcement 20. In addition, these housings also prevent the forming of overly substantial thicknesses of the strap with respect to the reinforcement.

It is also noted that the lower ends of the arms 18 overlap the heel stiffener 20 and the reinforcement strip 15 and are thereby cemented to the heel stiffener 20 and the reinforcement strip is, and consequently achieve a “correct” junction of these two elements. The strap 17 is cemented to the upper so as to pre-stress the rear wall 21 of the heel reinforcement toward the front. The sole 30 is then positioned and cemented to, the upper/insole/heel stiffener assembly thus constituted.

Such a manufacturing process allows a uniformity in the manufacture, while maintaining low cost due to the fact that the heel stiffener is molded. However, it can be used for other sizes since it is independent of the wear sole 30. The complexity and number of molds necessary is therefore reduced.

In practice, this type of heel stiffener 20 can be provided for at least two different shoe sizes. The aesthetic problem of possible junctions of the heel stiffener 20 and the front reinforcement strip 15 are also resolved by the overlapping covering of the connecting zone of these two elements by the strap 17.

In addition, repair is facilitated since it suffices to remove and replace the sole 30 without risking damaging the heel portion as in previously known constructions.

The present invention is not limited to the particular embodiments described hereinabove by way of non-limiting examples, but encompasses all similar or equivalent embodiments.

What is claimed is:

1. An article of footwear comprising:
a flexible upper;
a tightening arrangement provided on the upper to tighten the foot within the upper;
the upper comprising a molded heel stiffener;
an outer sole.

2. An article of footwear according to claim 1, wherein
said heel stiffener comprises a rear wall and a base extending substantially up to an area of a front limit of a plantar arch of a wearer.

3. An article of footwear according to claim 2, wherein:
said heel stiffener comprises two lateral edges adapted to protect rear lateral edges of the upper.

4. An article of footwear according to claim 3, wherein:
said heel stiffener comprises a pivoting recess at a junction of said lateral edges and said rear wall.

5. An article of footwear according to claim 1, wherein:
said heel stiffener includes housings for positioning and adhesively securing at least one strap or said outer sole.

6. An article of footwear according to claim 1, wherein:
said heel stiffener is made of rubber.

7. An article of footwear according to claim 1, wherein:
said outer sole comprises two portions with different thicknesses.

8. A method for assembling an article of footwear, said method comprising:
assembling the upper to an insole;
pressing and cementing to the upper a molded heel stiffener;
pressing and cementing to the front of the upper a front protective strip;
pressing and cementing to the rear of the upper a strap overlapping the heel stiffener and the front protective strip,
pressing and cementing the outer sole.
